SEQUENCE LISTING (12 sequences)

SEQ ID NO: 1 - DNA, 5255 bp, Artificial Sequence
  Description of Artificial Sequence Synthetic Plasmid phA-3xHA
attagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattcgcccttcaggcggtacagcctggagacaattgatggtttgcagctagaagacagtaaatctagcaatggagcccatatatgaggagtacctagcaaatcatggaacaatagtaaaaccatattactggctaagcttctctctcgattgctctaattgtccttaccatattcgaacaggtgaagaagcaagagtttccctcacagaattttgtcagatttttggattcccttatgggacaacatttcctcaaacaaaacacctcacattttatgaactaaaaacttcttctggtagcctggtgcaaaagggccatgctagcagttgcactgggaattatatccatccagaatcaatgctctttgaaatgaatggttatcttgactcagccatatacaataatgacagcatcaggcatatcattctgtattccaacaactccccttgtaatgaagctaaccactgctgcatcagcaaaatgtataatttcctgattacgtatccaggcatcactcttagtatttatttttctcagctctatcatactgagatggactttcctgcctcagcatggaaccgcgaagctctccggagcctggccagcttatggccgcgggttgttttgagtccaataagtggtgggatctggcattctgttctccacagctttataagtggtgtctcaggatcacatgtttttcagcccattttaactgggagagcactggctgacaggcacaacgcatatgaaatcaatgccataacaggcgtaaaaccttacttcactgatgttcttctccagacaaaaaggaatccaaacacaaaagctcaggaggctttagagagctaccccttaaacaatgcctttcctggacagttttttcaaatgccgagtggacaactccaacccaacctacctccagacctcagggctcctgttgtttttgtgctagtgcctctcagggacttaccaccaatgcatatgggccaaaacccaaataaacccaggaatatcgtaaggcacttaaatatgcctcaaatgtcattccaggaaaccaaggaccttggaaggcttcccactggaaggtcagtggagatagtggaaatcacagaacagtttgcaagtagcaaggaggcagatgaaaagaagaagaagaaagggaagaaaggggatccaccggtcgccaccatgtacccctacgacgtgcccgactacgccggctacccctacgacgtgcccgactacgccggctacccctacgacgtgcccgactacgccggctaactgagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgc

SEQ ID NO: 2 - protein, 405 aa, Artificial Sequence [sequence omitted]

SEQ ID NO: 3 - DNA, 6138 bp, Artificial Sequence
  Description of Artificial Sequence Synthetic Plasmid pHA-hA4-cDNA3.1Zeo(+)
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccctagcaatgggatatccatacgatgttccagattacgctgagcccatatatgaggagtacctagcaaatcatggaacaatagtaaaaccatattactggctaagcttctctctcgattgctctaattgtccttaccatattcgaacaggtgaagaagcaagagtttccctcacagaattttgtcagatttttggattcccttatgggacaacatttcctcaaacaaaacacctcacattttatgaactaaaaacttcttctggtagcctggtgcaaaagggccatgctagcagttgcactgggaattatatccatccagaatcaatgctctttgaaatgaatggttatcttgactcagccatatacaataatgacagcatcaggcatatcattctgtattccaacaactccccttgtaatgaagctaaccactgctgcatcagcaaaatgtataatttcctgattacgtatccaggcatcactcttagtatttatttttctcagctctatcatactgagatggactttcctgcctcagcatggaaccgcgaagctctccggagcctggccagcttatggccgcgggttgttttgagtccaataagtggtgggatctggcattctgttctccacagctttataagtggtgtctcaggatcacatgtttttcagcccattttaactgggagagcactggctgacaggcacaacgcatatgaaatcaatgccataacaggcgtaaaaccttacttcactgatgttcttctccagacaaaaaggaatccaaacacaaaagctcaggaggctttagagagctaccccttaaacaatgcctttcctggacagttttttcaaatgccgagtggacaactccaacccaacctacctccagacctcagggctcctgttgtttttgtgctagtgcctctcagggacttaccaccaatgcatatgggccaaaacccaaataaacccaggaatatcgtaaggcacttaaatatgcctcaaatgtcattccaggaaaccaaggaccttggaaggcttcccactggaaggtcagtggagatagtggaaatcacagaacagtttgcaagtagcaaggaggcagatgaaaagaagaagaagaaagggaagaaataagaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 4 - protein, 377 aa, Artificial Sequence
  Description of Artificial Sequence Synthetic Plasmid pHA-hA4-cDNA3.1Zeo(+)
MGYPYDVPDYAEPIYEEYLANHGTIVKPYYWLSFSLDCSNCPYHIRTGEEARVSLTEFCQIFGFPYGTTFPQTKHLTFYELKTSSGSLVQKGHASSCTGNYIHPESMLFEMNGYLDSAIYNNDSIRHIILYSNNSPCNEANHCCISKMYNFLITYPGITLSIYFSQLYHTEMDFPASAWNREALRSLASLWPRVVLSPISGGIWHSVLHSFISGVSGSHVFQPILTGRALADRHNAYEINAITGVKPYFTDVLLQTKRNPNTKAQEALESYPLNNAFPGQFFQMPSGQLQPNLPPDLRAPVVFVLVPLRDLPPMHMGQNPNKPRNIVRHLNMPQMSFQETKDLGRLPTGRSVEIVEITEQFASSKEADEKKKKKGKK

SEQ ID NO: 5 - DNA, 6108 bp, Artificial Sequence
  Description of Artificial Sequence Synthetic Plasmid phA4-cDNA3.1Zeo(+)
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccctagcaatggagcccatatatgaggagtacctagcaaatcatggaacaatagtaaaaccatattactggctaagcttctctctcgattgctctaattgtccttaccatattcgaacaggtgaagaagcaagagtttccctcacagaattttgtcagatttttggattcccttatgggacaacatttcctcaaacaaaacacctcacattttatgaactaaaaacttcttctggtagcctggtgcaaaagggccatgctagcagttgcactgggaattatatccatccagaatcaatgctctttgaaatgaatggttatcttgactcagccatatacaataatgacagcatcaggcatatcattctgtattccaacaactccccttgtaatgaagctaaccactgctgcatcagcaaaatgtataatttcctgattacgtatccaggcatcactcttagtatttatttttctcagctctatcatactgagatggactttcctgcctcagcatggaaccgcgaagctctccggagcctggccagcttatggccgcgggttgttttgagtccaataagtggtgggatctggcattctgttctccacagctttataagtggtgtctcaggatcacatgtttttcagcccattttaactgggagagcactggctgacaggcacaacgcatatgaaatcaatgccataacaggcgtaaaaccttacttcactgatgttcttctccagacaaaaaggaatccaaacacaaaagctcaggaggctttagagagctaccccttaaacaatgcctttcctggacagttttttcaaatgccgagtggacaactccaacccaacctacctccagacctcagggctcctgttgtttttgtgctagtgcctctcagggacttaccaccaatgcatatgggccaaaacccaaataaacccaggaatatcgtaaggcacttaaatatgcctcaaatgtcattccaggaaaccaaggaccttggaaggcttcccactggaaggtcagtggagatagtggaaatcacagaacagtttgcaagtagcaaggaggcagatgaaaagaagaagaagaaagggaagaaataagaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 6 - protein, 367 aa, Artificial Sequence
  Description of Artificial Sequence Synthetic Plasmid phA4-cDNA3.1Zeo(+)
MEPIYEEYLANHGTIVKPYYWLSFSLDCSNCPYHIRTGEEARVSLTEFCQIFGFPYGTTFPQTKHLTFYELKTSSGSLVQKGHASSCTGNYIHPESMLFEMNGYLDSAIYNNDSIRHIILYSNNSPCNEANHCCISKMYNFLITYPGITLSIYFSQLYHTEMDFPASAWNREALRSLASLWPRVVLSPISGGIWHSVLHSFISGVSGSHVFQPILTGRALADRHNAYEINAITGVKPYFTDVLLQTKRNPNTKAQEALESYPLNNAFPGQFFQMPSGQLQPNLPPDLRAPVVFVLVPLRDLPPMHMGQNPNKPRNIVRHLNMPQMSFQETKDLGRLPTGRSVEIVEITEQFASSKEADEKKKKKGKK

SEQ ID NO: 7 - DNA, 32 bp, Artificial Sequence
  Description of Artificial Sequence Forward primer
caggcggtaccagcctggagacaaattgatgg

SEQ ID NO: 8 - DNA, 57 bp, Artificial Sequence
  Description of Artificial Sequence Reverse primer
gacttggatcccctttcttccctttcttcttcttcttttcatctgcctccttgctac

SEQ ID NO: 9 - DNA, 68 bp, Artificial Sequence
  Description of Artificial Sequence Forward primer
cggatccctagcaatgggatatccatacgatgttccagattacgctgagcccatatatgaggagtacc

SEQ ID NO: 10 - DNA, 32 bp, Artificial Sequence
  Description of Artificial Sequence Reverse primer
gaattctttatttcttccctttcttcttcttc

SEQ ID NO: 11 - DNA, 29 bp, Artificial Sequence
  Description of Artificial Sequence Forward primer
cggatccctagcaatggagcccatatatg

SEQ ID NO: 12 - DNA, 32 bp, Artificial Sequence; identical to SEQ ID NO: 10
  Description of Artificial Sequence Reverse primer

(19) United States
(12) Patent Application Publication
(10) Pub. No.: US 2011/0129490 A1
(43) Pub. Date: Jun. 2, 2011

(54) MODULATION OF PRODUCTION OF RETROVIRUSES BY APOBEC4

(75) Inventors: Mario Perkovic, Karlsruhe (DE); Carsten Munk, Frankfurt (DE); Klaus Cichutek, Langen (DE); Daniela Marino, Frankfurt (DE)

(73) Assignee: BUNDERSREPUBLIK DEUTSCHLAND

(21) Appl. No.: 12/665,784

(22) PCT Filed: Jun. 26, 2008
(86) PCT No.: PCT/EP2008/005230

§ 371 Date: Jun. 15, 2010

(30) Foreign Application Priority Data
Jun. 27, 2007 (EP) 07012583.6

(51) Int. Cl. A61K 39/00; C12N 9/78; C12N 5/10; C07H 21/00; C12N 15/63; C07H 21/02; A61K 31/7088; A61K 35/12; C12Q 1/34; C12Q 1/68; G01N 33/68; C12Q 1/70; A61P 37/04; A61P 31/14

(52) U.S. Cl. 424/193.1; 435/227; 435/325; 536/23.2; 435/320.1; 424/184.1; 435/375; 514/44 R; 424/93.21; 435/18; 435/6.18; 435/7.1; 435/5

(57) ABSTRACT

The invention relates to APOBEC4 proteins and N- or C-terminally modified APOBEC4 proteins for the modulation of the release of retroviruses or viral particles thereof from cells and various uses thereof. Expression of APOBEC4 or N-terminally stabilized APOBEC4 protein in a cell leads to an increased viral production, while expression of C-terminally modified APOBEC4 protein leads to a decrease in viral production.

### TECHNICAL FIELD

The invention relates to the modulation of the release of retroviruses or viral particles thereof from cells by APOBEC4 or a modified APOBEC4 and various uses thereof.

### BACKGROUND ART

The apolipoprotein B mRNA-editing enzyme catalytic polypeptide-like 4 (APOBEC4) family is a member of the vertebrate specific AID/APOBEC superfamily of RNA/DNA editing cytidine deaminases (ROGOZIN, I B, et al. APOBEC4, a new member of the AID/APOBEC family of polynucleotide (deoxy)cytidine deaminases predicted by computational analysis. Cell Cycle. 2005, vol. 4, no. 9, p. 1281-5.) The AID/APOBEC protein superfamily contains five subfamilies (AID, APOBEC1, APOBEC2, APOBEC3 and APOBEC4) and includes several members with the capability of deaminating cytosine to uracil in single-stranded polynucleotides, while fulfilling diverse physiological functions (CONTICELLO, S G, et al. Evolution of the AID/APOBEC family of polynucleotide (deoxy)cytidine deaminases. Mol Biol Evol. 2005, vol. 22, no. 367, p. 77).

In mammals, expression of APOBEC4 is upregulated in testis which suggests the possibility that it is an editing enzyme for mRNAs involved in spermatogenesis. However, the exact physiological function(s) of APOBEC4 are still unknown. The nucleic acid and amino acid sequence for APOBEC4 is known and available for various species, inter alia, human (GenBank accession number NM_{—}203454, GI 44888831), mouse (GenBank accession number NC_{—}000067, GI 38073497), or rat (GenBank accession number NM_{—}001017492, GI 62945336). APOBEC4 is a protein of about 370 amino acids and 41000 Da (human APOBEC4: 367 amino acids; 41581 Da) and comprises a zinc finger domain and, in most species, a C-terminal poly-lysine tail.

### DISCLOSURE OF INVENTION

The present invention is based on the discovery that expression of the APOBEC4 protein or a modified APOBEC4 protein in a cell producing a retrovirus leads to an increased or decreased production of retroviruses or viral particles thereof from said cell. Specifically, stabilization of the protein by modification of the N-terminus, inter alia, by addition of an amino acid sequence, for example, the V5 tag, the c-myc tag or the HA tag, leads to an increased production of retroviruses or viral particles thereof from said cell. Modification of the C-terminus to decrease or inhibit binding to the C-terminal poly-lysine tail of APOBEC4, inter alia, by addition of at least one V5 tag, c-myc tag or HA tag or deletion or modification by mutation of the C-terminal poly-lysine tail of APOBEC4, leads to a decreased production of retroviruses or viral particles thereof from said cell.

A first aspect of the invention is a modified APOBEC4 protein characterized in that the N-terminus is modified to stabilize the APOBEC4 protein or the C-terminus is modified to decrease or inhibit binding to the C-terminal poly-lysine tail of APOBEC4. Preferably, the modification of the N-terminus comprises addition of at least one amino acid sequence selected from the group consisting of the V5 tag, the c-myc tag and the HA tag or an amino acid sequence homologous thereto. More preferably, the at least one amino acid sequence is HA or at least 85%, 87%, 90%, 95%, 97%, 99%, or 99.5% identical thereto. In another preferred embodiment the modification of the C-terminus comprises deletion or substitution of at least one amino acid of the poly-lysine tail or comprises addition of at least one amino acid sequence selected from the group consisting of the V5 tag, the c-myc tag and the HA tag or an amino acid sequence homologous thereto. More preferably, the at least one amino acid sequence is HA, 3 HA or at least 85%, 87%, 90%, 95%, 97%, 99%, or 99.5% identical thereto.

A further aspect of the invention is a cell expressing the C- or N-terminally modified APOBEC4 protein of the present invention. Preferably, the cell further produces a retrovirus. More preferably, the retrovirus or lentivirus is selected from the group consisting of HIV, HIV-1, HIV-2, SIV, SIVagm, SIVmac, FIV, EIAV, spuma virus (foamy virus) and MLV. Even more preferably, said retrovirus or lentivirus is a replication deficient virus. In a further preferred embodiment the cell is selected from the group consisting of a human cell, a HeLa cell, a 293T cell, a stem cell, a T-helper cell, a macrophage or progenitors or cell lines thereof.

Another aspect of the present invention is a nucleic acid comprising the nucleic acid sequence coding for the modified APOBEC4 protein of the present invention or the complement thereof. In a preferred embodiment the nucleic acid comprises a nucleic acid coding for the APOBEC4 protein of the present invention or complement thereof, operably linked to a promoter. In a more preferred embodiment the nucleic acid is a vector for gene therapy. In a further more preferred embodiment the promoter is the CMV promoter.

Another aspect of the present invention is the use of the nucleic acid sequence or cell of the present invention for the production of a medicament. In a preferred embodiment the nucleic acid is a vector for gene therapy and/or a nucleic acid comprising the nucleic acid coding for the modified APOBEC4 protein of the present invention operably linked to a promoter, preferably the CMV promoter. In a further preferred embodiment the cell expresses the modified APOBEC4 protein of the present invention. In a further preferred embodiment the cell expresses the modified APOBEC4 protein of the present invention and furthermore produces a retrovirus or virus particles thereof, preferably a replication deficient retrovirus, more preferably a replication deficient virus selected from the group consisting of HIV, SIV, MLV, FIV, EIAV, HIV-1, HIV-2, SIVagm, SIVmac, spuma virus or virus particles thereof. In a further aspect the medicament is directed to the treatment of or vaccination against HIV, SIV, MLV, FIV, EIAV, HIV-1, HIV-2, spuma virus, SIVagm and SIVmac.

Another aspect of the invention is the use of a viral particle of the invention for the preparation of a medicament, preferably for the preparation of a medicament for the treatment of viral infections. In a preferred embodiment, the viral particles comprise a C-terminally modified APOBEC4 protein of the invention, preferably the APOBEC4-HA or the APOBEC4-3×HA protein. In another preferred embodiment the viral particles are based on a virus selected from the group consisting of HIV, SIV, MLV, FIV, EIAV, HIV-1, HIV-2, SIVagm, SIVmac, spuma virus, preferably HIV-1. In a further preferred embodiment, the viral particles are pseudotyped viral particles. In another embodiment of the present invention the viral particle encodes sh/siRNA which targets the endogenous APOBEC4 and further encodes a codon optimized therapeutic APOBEC4, for example APOBEC-3×HA or APOBEC-HA, which is not affected by the sh/siRNA molecules.

Another aspect of the present invention is a vaccine comprising the cell of the present invention and an acceptable pharmaceutical carrier. In a preferred embodiment, the cell expresses the modified APOBEC4 protein of the present invention, wherein the APOBEC4 protein is modified at the N-terminus to stabilize the APOBEC4 protein and further produces a retrovirus or virus particles thereof, preferably a replication deficient retrovirus or lentivirus. In a further preferred embodiment the cell is a stem cell or an autologous cell, a T-helper cell or a macrophage.

Another aspect of the present invention is a method for decreasing the production of retroviruses or viral particles thereof of a cell producing a retrovirus or viral particles thereof, the method comprising expressing a modified APOBEC4 protein of the present invention in said cell, wherein the C-terminus of the APOBEC4 protein is modified to decrease or inhibit binding to the C-terminal poly-lysine tail of APOBEC4. In a preferred embodiment, the production of retroviruses or viral particles thereof is decreased by at least 20%, 30%, 40%, 50%, 60%, 70%, 80%, 85%, 90%, 95%, 97% or 99%.

A further aspect of the present invention is a method for producing retroviral particles, the method comprising cultivating a cell according to the present invention, wherein the cell produces a replication deficient retrovirus or virus particles of a retrovirus and furthermore expresses a modified APOBEC4 protein of the present invention, wherein the N-terminus of the APOBEC4 protein is modified to stabilize the APOBEC4 protein. In a preferred embodiment, the N-terminus of the APOBEC4 protein is modified by addition of a HA tag. In a further preferred embodiment the cell is a 293T cell or a HeLa cell co-transfected with an HA-APOBEC4 fusion construct and a HIV-1 expression plasmid.

A further aspect of the present invention is a method for screening for antiviral compounds, the method comprising the steps of contacting a cell expressing an APOBEC4 protein with a test compound, determining whether the test compound affects APOBEC4 transcription and/or expression of said cell. In a preferred embodiment the APOBEC4 transcription and/or expression of a first cell in the presence of the test compound is compared to the APOBEC4 transcription and/or expression of a second cell not contacted with the test compound, wherein a decrease in APOBEC4 transcription and/or expression of the first cell indicates that the test compound is a potential antiviral compound.

In a preferred embodiment, HeLa or 293T cells are used in the screening method. In a further preferred embodiment the amount of APOBEC4 transcript and/or APOBEC4 protein is determined by RT-PCR, Northern blotting and western blotting, respectively.

A further aspect of the invention is a method for screening for antiviral compounds, the method comprising the steps of contacting a cell expressing a retrovirus and APOBEC4 with a test compound and determining the titer of the retrovirus in the presence and the absence of the test compound, wherein a decrease in the titer indicates that the candidate compound interferes with the assembly of the retrovirus by interaction with the APOBEC4 protein and thus is a potential antiretroviral drug.

Further aspects of the invention are molecules—and pharmaceutics based on such molecules—directed to and interfering with the APOBEC4 mRNA. Preferably said molecules are siRNA or shRNA molecules directed against APOBEC4 mRNA. Preferably, such molecules are used for the manufacture of a medicament. Even more preferably, said molecules are used for the preparation of a medicament for the treatment of viral infections.

Additional embodiments of the present invention are described in the claims and the detailed description of the invention.

### BRIEF DESCRIPTION OF FIGURES IN THE DRAWINGS

FIG. 1 is a schematic structural overview over the human APOBEC4 protein and consequences of modification of the C-terminus, exemplarily demonstrated by an HA-tag.

FIG. 2 depicts the vector maps of plasmids phA4-3×HA (FIG. 2A), phHA-A4cDNA3.1Zeo(+) (FIG. 2B) and phA4cDNA3.1Zeo(+) (FIG. 2C), respectively.

FIG. 3 shows the results of the western blotting of 293T cells co-transfected with HIV-1 NL4-3 and an expression plasmid coding for an unmodified APOBEC4, HA-APOBEC4 and APOBEC4-3×HA, respectively.

FIG. 4 shows the results of the western blotting of T293 cells co-transfected an expression plasmid encoding the reporter virus HIV-1 NL-luc R-E- and the expression plasmid coding for the unmodified APOBEC4, HA-APOBEC4 and APOBEC4-3×HA, respectively.

FIG. 5 shows the results of the reporter virus assays, wherein co-transfection of the cells with the reporter virus HIV-1 NL-luc R-E- and the expression plasmid coding for the unmodified APOBEC4, HA-APOBEC4 and APOBEC4-3HAare shown.

### DETAILED DESCRIPTION

### Definitions

“APOBEC4”, as used in the present invention refers to the apolipoprotein B mRNA-editing enzyme catalytic polypeptide-like 4 protein or gene coding for said protein. The modified APOBEC4 proteins of the present invention that contain additional amino acid sequences, for example a tag like the HA tag, are designated as HA-APOBEC4 if the tag is attached to the N-terminus of the APOBEC4 protein and APOBEC4-HA if the tag is attached to the C-terminus of the APOBEC4 protein. Thus, V5-APOBEC4 and APOBE4-V5 designate a fusion protein, wherein the V5 tag is fused to the N- and C-terminus of the APOBEC4 protein, respectively. As a further example, APOBEC4-3×HA designates a fusion protein of APOBEC4 and three HA tags sequentially fused to the C-terminus of APOBEC4. Nucleic acids or plasmids are designated in the same manner.

The “viruses” of the present invention encompass all retroviruses, preferably lentiviruses, more preferably HIV, SIV, FIV, EIAV, MLV, spuma virus, even more preferably HIV-1, HIV-2, SIVagm, SIVmac, and most preferably HIV-1. Furthermore, mutated and/or truncated viruses derived from the above retroviruses are encompassed by the present invention. A subset of such mutated/truncated retroviruses are the so called “vector particles” or “viral particles”, which refer to replication deficient retroviruses. Thus, the retroviral vector particles of the present invention are retroviral vectors that may contain an RNA which, upon infection of a host or target cell, is reverse-transcribed and inserted into the genome of said cell. This RNA may contain a heterologous gene, for example the luciferase reporter gene of Photinus pyralis as in the NL-luc R-E- reporter virus described infra. However, these vector particles only contain an incomplete genome of the lentivirus from which they are derived. In general, the RNA molecule of the vector particle does not comprise the genetic information of the gag, env, and/or pot genes itself, which is a known minimal requirement for successful replication of a retrovirus. As a result, the vector particles are replication deficient, i.e. they are not able to transduce a host or target cell with the genetic information required for their own replication.

A vector particle thus comprises a minimum of the Gag, Pol, and Env proteins and an RNA molecule (which can be an expression vector). The RNA molecule is derived from the genome of the retrovirus but does not comprise at least one of the gag, env, or pot genes itself. However, to produce a safe and efficient vector particle, in general all three of said genes are lacking, as well as any other nonessential genes. In case of HIV-1, such unnecessary genes of the HIV-1 genome that can be deleted from the RNA molecule include tat, vif, vpr, vpu and nef.

The RNA molecule may still comprise all elements, for example, the psi element and LTRs, of the retroviral genome which are required for an effective packaging of the RNA into the resulting vector particles. In the case of an expression vector, the RNA molecule preferably comprises a further gene (usually heterologous) that is under the control of a suitable promoter, for example, the CMV promoter, and is thus expressed upon integration of the gene into the genome of the host or target cell.

Therefore, one example of an RNA molecule that may be used to generate a retroviral vector particle is based on the HIV-1 genome and comprises the LTRs, the psi element and the CMV promoter followed by the gene to be transduced, for example, a reporter gene such as the gene for a GFP protein or the luciferase reporter gene of Photinus pyralis. On expression of such reporter genes, the titers or the amount of the generated vector particles can conveniently be determined by standard assays. The gag, env, pol, tat, vif, vpr, vpu and/or nef genes of HIV-1 are removed or expression of their gene products is prevented by, for example, frame shift mutation(s).

Another example for a heterologous gene comprised by the RNA molecule is the gene coding for the (modified) APOBEC4 protein of the present invention. Thus, the present invention provides vector particles that are based on and display the tropism of a specific retrovirus, for example HIV-1, and for which treatment is sought and further comprise a gene coding for a modified APOBEC4 protein of the present invention that decreases production of the retrovirus. Such vector particles will transfect the same cells, possibly infected by HIV-1, and subsequently decrease production of HIV-1 by said cells.

The minimal requirements for a lentivirus vector based on HIV-1 have been described by KIM, V N, et al., Minimal requirement for a lentivirus vector based on human immunodeficiency virus type 1. J. Virol. 1998, vol. 72, no. 1, p. 811-6.

The above-described RNA molecule together with the gag, pol and env proteins, that may be provided in trans by the packaging cell line, are then assembled into the vector particles or vector particles, which will be able to efficiently infect their target or host cells, reverse-transcribe the RNA molecule that may comprise a heterologous gene under the control of a promoter, for example the CMV promoter, and integrate said genetic information into the genome of the target/host cells. However, as the genetic information for the gag, pol and/or env proteins is not present on the transduced RNA molecule, the vector particles or vector particles will be replication deficient, i.e. no new generation of said vector particles will thus be generated by the transduced cell.

The term “stabilization”, as used in the present invention refers to an inhibition or decrease of protein degradation or an inhibited or decreased transport of the protein from, for example, the cytosol into another cellular compartment or out of the cell, thus leading to an increased availability of the protein within the cell, for example, in the cytosol. The skilled person is aware of various ways to achieve stabilization of a protein, inter alia, by modification of the protein through amino acid substitutions, additions or deletions. A preferred modification to achieve stabilization is the N-terminal addition of one or more amino acid sequences selected from the group consisting of the V5 tag, the c-myc tag and the HA tag or an amino acid sequence homologous thereto.

The person skilled in the art will readily be able to introduce mutations as, for example, additions and deletions, into a given nucleic acid or amino acid sequence. Such known methodologies are, for example, disclosed in Sambrook et al. (1989).

The invention particularly includes nucleic acids sequences or homologs thereof, or unique fragments thereof. In the present invention, the sequence of a nucleic acid molecule that encodes a resulting protein is considered homologous to a second nucleic acid molecule if the nucleotide sequence of the first nucleic acid molecule is at least about 70% identical, preferably at least about 80% identical, and more preferably at least about 85%, 90%, 95% or 99% identical to the sequence of the second nucleic acid molecule. Alternatively, homology between two nucleic acid sequences may be readily determined using the known BLASTN algorithm (ALTSCHUL, S F, et al. Basic local alignment search tool. J Mol. Biol. 1990, vol. 215, no. 3, p. 403-10) with default settings. As a further example, another known test for ascertaining the homology of two nucleic acid sequences is whether they hybridize under normal hybridization conditions, preferably under stringent hybridization conditions.

Using the nucleic acid sequences disclosed herein, the skilled person can further design nucleic acid structures having particularly desired functions in various types of applications. For example, the artisan can construct oligonucleotides or polynucleotides for use as primers in nucleic acid amplification procedures, such as the polymerase chain reaction (PCR), ligase chain reaction (LCR), Repair Chain Reaction (RCR), PCR oligonucleotide ligation assay (PCR-OLA), and the like. Oligonucleotides useful as probes in hybridization studies, such as in situ hybridization, can also be constructed. Numerous methods for labeling such probes with radioisotopes, fluorescent tags, enzymes, and binding moieties (e.g., biotin) are known, thus the probes of the present invention can be adapted for detectability in a straightforward manner.

The protein of the present invention further includes functional homologs. A protein is considered a functional homolog of another protein for a particular function, if the homolog has a similar function as the original protein. The homolog can be, for example, a fragment of the protein, or a substitution, addition, or deletion mutant of the protein.

Determining whether two amino acid sequences are substantially homologous is typically based on FASTA searches in accordance with Pearson et al., PERASON, W R, et al. Improved tools for biological sequence comparison. Proc Natl Acad Sci USA. 1988, vol. 85, no. 8, p. 2444-8. For example, the amino acid sequence of a first protein is considered to be homologous to that of a second protein if the amino acid sequence of the first protein shares at least about 70% amino acid sequence identity, preferably at least about 80% identity, and more preferably at least about 85%, 90%, 95%, 97%, 99% or 99.5%, most preferably at least 99% identity, with the sequence of the second protein.

The term “pseudotyped”, as used in the present invention, refers to a vector particle bearing envelope glycoproteins derived from other viruses having envelopes. The host range of the retroviruses or lentiviruses of the present invention can thus be expanded or altered depending on the type of cell surface receptor used by the glycoprotein.

The gag, pol and env proteins needed to assemble the vector particle are provided in trans by means of a packaging cell line, for example, HEK-293T. This is usually accomplished by transfection of the packaging cell line with one or more plasmids containing the gag, pol and env genes. For the generation of pseudotyped vectors, the env gene, originally derived from the same retrovirus as the gag and pol genes and as the RNA molecule or expression vector, is exchanged for the envelope protein(s) of a different enveloped virus. As an example, the G protein of VSV is used.

Thus, an exemplary pseudotyped vector particle based on the HIV-1 retrovirus comprises the (1) HIV-1 Gag and Pol proteins, (2) an RNA molecule derived from the HIV-1 genome that may be used to generate a retroviral vector particle based on the HIV-1 genome lacking the gag, env, pol, tat, vif, vpr, vpu and nef genes, but still comprising the LTRs, the psi element and a CMV promoter followed by the gene to be transduced, for example, a gene for the GFP protein, and (3) the G protein of VSV. The resulting pseudotyped vector particle will thus display the tropism of VSV.

A further example is the reporter virus NL-luc R-E- that is derived from the HIV-1 variant NL4-3 and features a deletion of the vpr, env and nef genes. Furthermore, NL-luc R-E- carries the luciferase reporter gene of Photinus pyralis and is pseudotyped with the VSV-G protein.

A virus or vector particle “derived from”, for example, HIV-1, as used in the present invention, refers to a virus or vector particle in which the genetic information for the RNA and/or the Gag and Pol proteins comprised by the virus or vector particle originally stems from the original virus, in the above case, HIV-1. As described above, the original retroviral genome can comprise mutations, such as deletions, frame shift mutations and insertions.

The term “tag” as used in the present invention, refers to a short amino acid sequence which serves as an epitope for immunological detection of protein of interest. Tags used in the present invention are HA, V5 and c-myc.

### Taxonomy

Viruses of the taxonomic family of the Retroviridae share, as a unifying feature, a positive stranded ssRNA genome (herein “ss(+)RNA”). During infection, said ss(+)RNA can not directly be used as a template (mRNA), thus the genetic information of this virus is first reverse-transcribed from RNA into DNA by a reverse transcriptase provided by the virus and subsequently integrated into the host genome. Thus, the term “retro” refers to the activity of reverse transcriptase and the transfer of genetic information from RNA to DNA. Presently, more than 570 individual viruses of the family have been identified.

One sub-family of Retroviridae comprises the Orthoretroviridae viruses, including the genera of the alpha-retroviruses with, for example, the Avian leukosis virus (ALV) species the gamma-retroviruses with, for example, the Murine leukemia virus (MLV) species or the Feline leukemia virus (FLV) or the delta-retroviruses with, for example, the Bovine leukemia virus (BLV) species or the Human T-lymphotropic virus (HTLV).

One additional genus of the Orthoretroviridae, known as the lentiviruses, has recently attracted much interest largely because of a well-known member, the Human immunodeficiency virus (HIV). However, the genus of lentiviruses comprises numerous other viruses. In addition to the HIV-1 or HIV-2 species, the Bovine immunodeficiency virus (BIV), the Feline immunodeficiency virus (FIV) and the Simian immunodeficiency virus (SIV) are all examples of frequent and extensive research efforts. A general overview of the retroviruses and lentiviruses, respectively can be found, for example, in Vigna et al. and Palu et al. (VIGNA, E, et al. Lentiviral vectors: excellent tools for experimental gene transfer and promising candidates for gene therapy. J Gene Med. 2000, vol. 2, no. 5, p. 308-16. PALU, G, et al. Progress with retroviral gene vectors. Rev Med Virol. 2000, vol. 10, no. 3, p. 185-202).

### Structural and Genetic Features of Retroviral Genomes

The genomes of known replication-competent retroviruses typically comprise the four coding domains (gag, pro, pol, env), wherein gag encodes a polypeptide (herein “Gag”) wherein the cleavage products comprise the major structural proteins of the virus core including Matrix (MA), capsid (CA), and nucleocapsid (NC). Pro encodes part of the polyprotein (Gag-Pro or Gag-Pro-Pol) wherein the cleavage products typically include protease (PR) and occasionally dUTPase (DU). Pol encodes part of the polyprotein (Gag-Pro-Pol), wherein the cleavage products typically include reverse transcriptase (RT) and integrase (IN) and, in some lentiviruses, dUTPase (DU). In spumaviruses, pol is expressed via a spliced mRNA as Pol-Pol polyprotein. Env encodes a polyprotein (Env) whose cleavage products SU (surface) and TM (transmembrane) comprise the structural proteins of the viral envelope.

Furthermore, retroviruses typically contain two long terminal repeats (LTRs) and a region of several hundred (−300-1800) base pairs composed of U3-R-U5 (5 to 3) which are located at both ends of the unintegrated and integrated (proviral) genome. Additionally, a psi element is generally present within the retroviral genome. The psi element is a cis-acting signal, located near the 5′ end of the genome and designates a packaging signal, which is of importance during virus assembly and leads to the incorporation of the viral RNA into the viral core. More complex retroviruses typically comprise additional regulatory genes. In case of HIV-2, these regulatory genes include ref, tat, vif, nef, vpr and vpx.

### Overview of the Function of APOBEC4 in Virus Production

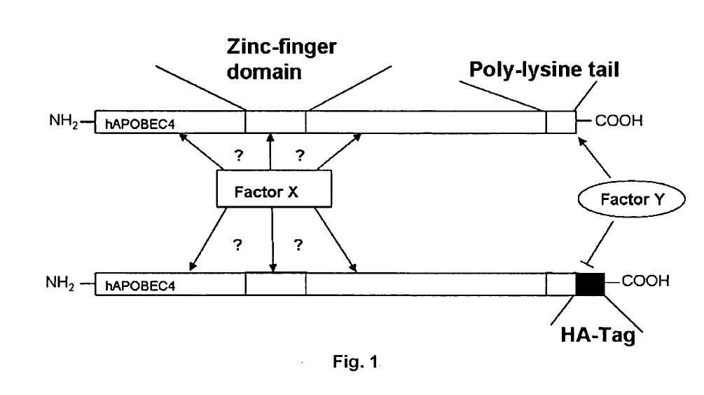

APOBEC4 is a eukaryotic protein and expressed primarily in mammalian testis which suggesting that it is an editing enzyme for mRNAs involved in spermatogenesis. As a non-limiting working hypothesis (see FIG. 1a), during viral replication a complex comprising APOBEC4 and at least two further and yet unknown factors (factor X and factor Y) is recruited and used by the replication machinery of the virus. As depicted in FIG. 1a, APOBEC4 comprises a zinc-finger domain (hpesmlfemngyldsaiynndsirhiilysnnspcneanhcc, aa 93-134) and a C-terminal poly-lysine tail (kkkkkgkk, aa 360-367), wherein the poly-lysine tail enables binding of factor Y, while factor X presumably binds to the zinc-finger domain. Furthermore, a surplus within the cell of the two factors, X and Y, compared to endogenous APOBEC4 is assumed.

The present invention is based on the surprising experimental finding that expression of the APOBEC4 protein in a cell producing a retrovirus leads to an increased or decreased production of retroviruses from said cell. Furthermore, this pro-viral effect directly correlated with the amount of plasmid DNA coding for the APOBEC4 protein used for the co-transfection and thus with the expressed APOBEC4 protein. This correlating pro-viral effect of APOBEC4 expression can be explained with an increased formation of the APOBEC4 complex recruited by the virus: Since there is a surplus of factor X and Y, the amount of APOBEC4 complexed with factor X and factor Y will only depend on the amount of APOBEC4 expressed by the cell (and thus depend on the amount of plasmid DNA used during transformation). An increase in virus production up to 10-fold was achieved using HA-APOBEC4 compared to the negative control (293T transfected only with provirus).

Proteins are constantly degraded and re-synthesized by the cell (a process known as protein turnover) or transported or translocated into various cellular compartments, for example, from the cytosol into the mitochondria. Consequently, if protein turnover is shifted, i.e. the protein degradation is prevented or slowed down, or transport of the protein from the cytosol into another cellular compartment is inhibited or slowed down, the protein will accumulate in the cytosol of the cell.

Surprisingly, the inventors discovered that stabilization/modification of the APOBEC4 protein by modification of the N-terminus, inter alia, by addition of an amino acid sequence, for example, the V5 tag, the c-myc tag or the HA tag, and expression of such a protein in a cell producing a retrovirus or viral particles thereof leads to an augmented increased production of retroviruses or viral particles by said cell compared to expression of unmodified APOBEC4 within that cell. The amount of viruses that were detectable by reverse transcriptase assays or western blotting within about 2 days after co-transfection in the supernatant of cells showed a 5-10 fold increase.

Another aspect of the present invention is based on the surprising discovery that modification of the C-terminus of the APOBEC4 protein to decrease or inhibit binding to the C-terminal poly-lysine tail of APOBEC4, inter alia, by addition of at least one V5 tag, c-myc tag or HA tag, and subsequent expression of such a modified APOBEC4 protein leads to a drastically decreased production (up to 95% or more) of retroviruses or viral particles thereof by said cell. Furthermore, this antiviral-viral effect directly correlated with the amount of plasmid DNA coding for the modified APOBEC4 protein used for the co-transfection and thus with the modified APOBEC4 protein expressed by the cell. This correlating anti-viral effect of the present invention can be explained by competition of expressed modified APOBEC4 with endogenous APOBEC4 for the factor X. The APOBEC4 protein modified at the C-terminus to prevent or decrease binding to the poly-lysine tail will bind factor X but will be unable to bind factor Y in order to form a functional APOBEC4 complexed with factor X and factor Y that is required by the virus for replication. Consequently, as the modified APOBEC4 competes for factor X, less of said factor will be available to the endogenous APOBEC4 and thus less functional APOBEC4 complex will be formed, leading to the observed decrease in virus production by the cell, i.e. the anti-viral effect.

### APOBEC4 Proteins and Constructs

The present invention makes use of APOBEC4 proteins of various species, preferably mammals, most preferably human APOBEC4. The preferred APOBEC4 proteins encompassed by the invention are APOBEC4 proteins modified according to the teachings disclosed herein.

Generally, modified APOBEC4 proteins are encompassed by the present invention that are stabilized, preferably by modification of, for example, the N-terminus, inter alia, by addition of an amino acid sequence, for example, the V5 tag, the c-myc tag or, preferably, the HA tag. Furthermore, amino acid sequences that are homologous to the V5 tag, the c-myc tag or, preferably, the HA tag, preferably, at least 90%, 95%, 97%, 99%, or 99.5% identical thereto, are also explicitly mentioned as amino acid sequences that may be used to modify the APOBEC4 protein according to the present invention. Thus, the present invention encompasses all APOBEC4 proteins that are stabilized by any means known to the skilled person. Furthermore, APOBEC4 proteins that are modified to inhibit or decrease binding of at least one factor required to form the APOBEC4 complex that is recruited during viral replication are encompassed by the present application. The skilled person is aware of a multitude of means to modify a protein to inhibit or decrease binding of factors thereto. Preferred APOBEC4 proteins of the invention encompass APOBEC4 proteins modified at the C-terminus by deletion or substitution of at least one amino acid of the poly-lysine tail or addition of at least one amino acid sequence selected from the group consisting of the V5 tag, the c-myc tag and the HA tag or an amino acid sequence homologous thereto. Furthermore, amino acid sequences that are homologous to the V5 tag, the c-myc tag or, preferably, the HA tag, preferably, at least 90%, 95%, 97%, 99%, or 99.5% identical thereto, are also explicitly mentioned as amino acid sequences that may be used to modify the APOBEC4 protein according to the present invention. Further preferred APOBEC4 proteins of the invention are mutated within the zinc-finger domain, preferably, by E95A, C127A and C134A amino acid substitutions. The most preferred modified APOBEC4 proteins of the present invention are HA-APOBEC4, APOBEC4-HA, APOBEC-3×HA and APOBEC4 complete lacking the poly-lysine tail.

Nucleic acids coding for the APOBEC4 proteins of the present invention, for example plasmids constructed for the expression such proteins, are also encompassed. Furthermore, all proteins and nucleic acids homologous to the APOBEC4 protein or nucleic acids of the present invention are also encompassed. Preferably, the amino acid sequence of the homologous protein or the nucleic acid sequence of the homologous nucleic acid is at least 90%, 95%, 97%, 99%, or 99.5% identical to the APOBEC4 proteins or the nucleic acids of the present invention.

Given the working hypothesis outlined above, a multitude of additional modified APOBEC4 proteins and functional homologs thereof are obviously derivable for the skilled person. As a preferred example, KRR1 (GeneBank access number NP_{—}008974), the human HIV-1 rev binding protein 2 is given that likewise comprises a poly-lysine tail. Thus, KRR1-HA, HA-KRR1, KRR1-3×HA and KRR1 with a truncated or mutated poly-lysine tail are preferred embodiments. Furthermore, the poly-lysine tail of APOBEC4 itself, either in unmodified form or comprising amino acid substitutions is envisaged to be a possible functional homolog of APOBEC4 as it may compete for at least one of the factors required to form a functional APOBEC4 complex recruited during viral reproduction.

### Cells

While the present invention has exemplarily been described and studied in 293T cells and HeLa cells, it is contemplated that other cells may be used according to the teachings of the present invention. Preferably, the cells used in accordance with the present invention are human cells, macrophages, T cells, stem cells, autologous cells, as well as progenitor cells or cell lines thereof. Most preferably, the cells are 293T cells or HeLa cells.

### Medical Uses and Pharmaceutical Compositions

The constructs of the present invention can directly be used in a variety of medical applications or for the manufacture or medicaments.

In one aspect of the invention the N-terminally stabilized/modified APOBEC4 proteins of the invention can be expressed in a cell producing a retrovirus or viral particles thereof and be used to augment production of retroviruses or viral particles by said cell. Therefore, the present invention provides a method for increasing the production of retroviruses, inter alia, HIV, SIV, FIV, EIAV, MLV, HIV-1, HIV-2, SIVagm, SIVmac, spuma virus or viral particles thereof in a cell that can subsequently be isolated and used for purposes of vaccination against such retroviruses.

In another embodiment, the present invention can be used for gene therapy purposes, inter alia, to treat, inhibit or decrease production of a retrovirus in a mammal. In one embodiment, the viruses or viral particles of the present invention, or pharmaceutical preparations thereof, can be administered to a mammal, preferably, a human or a horse. If the viruses or viral particles exhibit the same tropism as the retrovirus that is to be treated then selective infection of the cells of the mammal which are the target cells of the retrovirus to be treated by the viruses or viral particles of the present invention will result. Upon cell entry the viruses or viral particles of the invention express the C-terminally modified APOBEC4 protein which subsequently leads to a decrease in viral production from said cell. As an example, if HIV-1 is to be treated viruses derived from HIV-1 and comprising the genetic information for a C-terminally modified APOBEC4 protein, preferably APOBEC4-3×HA, are administered to the mammal. As the administered viruses are derived from HIV-1 they exhibit the same tropism as an HIV-1 virus and will consequently infect the same cell-types, for example T cells. The infected cells will now express the C-terminally modified APOBEC4 protein, which will decrease production of HIV-1. It is evident to the skilled person that infections with any other retrovirus, including HIV, SIV, FIV, EIAV, MLV, HIV-2, spuma virus, SIVagm and SIVmac, can be treated in this manner.

In a further embodiment, the viruses or viral particles of the present invention harbouring the genetic information for C-terminally modified APOBEC4 protein according to the present invention are used to transduce retroviral target cells ex vivo and then reintroduce these into the mammal to be treated. In case of HIV the target cells are preferably T helper cells and/or macrophages or their progenitor cells.

In a further embodiment the transfer construct transduced into the target cells of the retrovirus by means of the viruses or viral particles of the present invention encodes sh/siRNA which targets the endogenous APOBEC4 and further encodes a codon optimized therapeutic APOBEC4, for example APOBEC-3×HA or APOBEC-HA, which is not affected by the sh/siRNA molecules. In a further embodiment the transfer construct of the present invention encodes an antibody which targets the endogenous APOBEC4 and may or may not encode a modified therapeutic APOBEC4 protein, which is not affected by the antibodies.

In a further aspect of the present invention endogenous APOBEC4 is downregulated, silenced or depleted in order to treat retroviral infections. As demonstrated by the present invention, a downregulation of APOBEC4 leads to a significant decrease in virus production and therefore treatment of retroviral infection. Thus, any means leading to a decrease in the presence of endogenous APOBEC4 are suitable to treat or control a retroviral infection, preferable an HIV infection. Downregulation or depletion of endogenous APOBEC4 can be achieved by a variety of means which are known to the skilled person in the art. As an example and preferred embodiment, endogonous APOBEC4 is downregulated by administration of siRNA or shRNA directed to the APOBEC4 mRNA, for example, the 3′ untranslated region of APOBEC4 mRNA. In a further example and preferred embodiment, endogenous APOBEC4 is depleted from the system of the infected individuum by administration of an antibody directed to APOBEC4.

Further methods to “switch off” a gene by gene silencing, e.g. by epigenetic means, are known to the skilled person. The skilled person is aware of the fact that the APOBEC4 gene may be regulated either at the transcriptional or post-transcriptional level.

In one embodiment of this aspect, the present invention is directed to a method of treatment of retroviral infections, comprising the step of downregulating or depleting of endogenous APOBEC4. In a preferred embodiment the downregulation is achieved by administering to the individuum at least one siRNA and/or at least one shRNA that is directed to the APOBEC4 mRNA. In a more preferred embodiment, the at least one antisense RNA has the nucleic acid sequence as defined in SEQ ID NOs: 13-16. In another preferred embodiment the depletion is achieved by administering to the individuum at least one antibody directed to APOBEC4. Combinations of antisense and antibody treatment are further encompassed by the present invention.

The present invention is further directed to at least one siRNA and/or at least one shRNA directed to the APOBEC4 mRNA and/or at least one antibody directed to APOBEC4 as a medicament. In a preferred embodiment, the at least one siRNA and/or at least one shRNA is directed to the 3′ untranslated region of APOBEC4 mRNA. In a further embodiment, the invention is directed to at least one siRNA and/or at least one shRNA directed to the APOBEC4 mRNA and/or at least one antibody directed to APOBEC4 for the treatment of retroviral infections. As is evident from the preceeding paragraphs, the at least one siRNA, shRNA and antibody, respectively can be used alone or in combination.

Pharmaceutical compositions based on the constructs or the siRNA, shRNA or antibodies of the present invention can be formulated in any conventional manner using one or more physiologically acceptable carriers or excipients. Thus, the viruses or viral particles, nucleic acids, proteins and compounds inhibiting the transcription and/or expression of APOBEC4 or the proper functioning of APOBEC4 of the present invention may be formulated for administration by, for example, injection, inhalation or insulation (either through the mouth or the nose) or by oral, buccal, parenteral or rectal administration.

The pharmaceutical compositions of the present invention can be formulated for a variety of modes of administration, including systemic, topical or localized administration. Techniques and formulations can be found in, for example, Remington's Pharmaceutical Sciences, Meade Publishing Co., Easton, Pa. For systemic administration, injection is preferred, including intramuscular, intravenous, intraperitoneal, and subcutaneous. For the purposes of injection, the pharmaceutical compositions of the present invention can be formulated in liquid solutions, preferably in physiologically compatible buffers, such as Hank's solution or Ringer's solution. In addition, the pharmaceutical compositions may be formulated in solid form and redissolved or suspended immediately prior to use. Lyophilized forms of the pharmaceutical composition are also suitable.

For oral administration, the pharmaceutical compositions of the present invention may take the form of, for example, tablets or capsules prepared by conventional means with pharmaceutically acceptable excipients such as binding agents (e.g. pregelatinized maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycolate); or wetting agents (e.g. sodium lauryl sulfate). The tablets can also be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. ationd oil, oily esters, ethyl alcohol or fractionated vegetable oils); and preservatives (e.g. methyl or propyl-p-hydroxybenzoates or sorbic acid). The preparations can also contain buffer salts, flavoring, coloring and sweetening agents as appropriate.

The pharmaceutical compositions can be formulated for parenteral administration by injection, e.g. by bolus injection or continuous infusion. Formulations for injection can be presented in a unit dosage form, e.g. in ampoules or in multi-dose containers, with an optionally added preservative. The pharmaceutical compositions can further be formulated as suspensions, solutions or emulsions in oily or aqueous vehicles, and may contain other agents including suspending, stabilizing and/or dispersing agents.

Additionally, the pharmaceutical compositions can also be formulated as a depot preparation. These long acting formulations can be administered by implantation (e.g. subcutaneously or intramuscularly) or by intramuscular injection. Thus, for example, the compounds may be formulated with suitable polymeric or hydrophobic materials (e.g. as an emulsion in an acceptable oil) or ion exchange resins, or as sparingly soluble derivatives, for example, as a sparingly soluble salt. Other suitable delivery systems include microspheres, which offer the possibility of local noninvasive delivery of drugs over an extended period of time. This technology can include microspheres having a precapillary size, which can be injected via a coronary catheter into any selected part of an organ without causing inflammation or ischemia. The administered therapeutic is then slowly released from the microspheres and absorbed by the surrounding cells present in the selected tissue.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, bile salts, and fusidic acid derivatives. In addition, detergents may be used to facilitate permeation. Transmucosal administration can occur using nasal sprays or suppositories.

### Screening Assays for Antiviral Compounds

The key role, APOBEC4 plays in retroviral reproduction makes it a prime target for antiretroviral drugs. Thus, APOBEC4 can be used in an assay to screen for compounds that inhibit APOBEC4 transcription and/or expression, and/or the function of APOBEC4 in retroviral reproduction.

As it was found that endogenous APOBEC4 is expressed in, inter alia, 293T cells or HeLa cells. Such cells could be used in a high throughput assay to screen for compounds that inhibit APOBEC4 transcription and/or expression. It is apparent to the skilled person that also cells or cell lines expressing exogenous APOBEC4 could be used in such an assay.

In one embodiment of the invention, the method for screening for antiviral compounds comprises the steps of contacting a cell expressing an APOBEC4 protein with a test compound and determining whether the test compound affects APOBEC4 transcription and/or expression of said cell, wherein a decrease in APOBEC4 transcription and/or expression indicates that the test compound is an antiviral compound. In a preferred embodiment the APOBEC4 transcription and/or expression of a first cell in the presence of the test compound is compared to the APOBEC4 transcription and/or expression of a second cell not contacted with the test compound, wherein a decrease in APOBEC4 transcription and/or expression of the first cell indicates that the test compound is a potential antiviral compound. Preferably, the cells used in the screening method are human cells, macrophages, T cells, stem cells, autologous cells, as well as progenitor cells or cell lines thereof. Most preferably, the cells are 293T cells or HeLa cells. Preferably, the APOBEC4 transcription and/or expression is determined by RT-PCR, Northern blotting and western blotting, respectively.

In a further embodiment the invention is directed to a method of screening for screening for antiviral compounds. Candidate compounds may initially be selected or chemically designed based on their predicted or determined binding to the APOBEC4 protein. The predicted binding of a compound, e.g. a protein, to APOBEC4 can be computed using docking algorithms known to the skilled person which produce many possible structures of a protein-protein or protein-compound complex and in most cases some of them resemble the correct structure within an r.m.s.d. of <3 Å. The BIOCOR binding analysis can be used to measure the binding of a given compound to APOBEC4. 293T cells are then infected with a retrovirus and the titer of the retrovirus is determined in the presence and the absence of one or more of the selected candidate compounds. A decrease in the titer indicates that the candidate compound interferes with the assembly of the retrovirus by interaction with the APOBEC4 protein and thus is a potential antiretroviral drug.

As a further example, siRNA or shRNA molecules directed against APOBEC4 mRNA are used as antiretroviral test compounds. It is expected that knock-down of APOBEC4 by such siRNA or shRNA molecules will greatly reduce the release of viruses by the infected cell. Therefore, siRNA or shRNA molecules may be used as antiretroviral drugs.

### EXAMPLES

### Generation of Plasmids

Plasmids for the expression of APOBEC4 and modified APOBEC4 were generated by PCR, starting from the APOBEC4 cDNA (GeneBank accession no NM_{—}203454) from human germ cells. The unmodified APOBEC4 protein and the modified APOBEC4 fusion protein comprising an N-terminal addition of a tag were expressed from the pcDNA3.1/Zeo(+) plasmid (Invitrogen). The modified APOBEC4 fusion protein comprising a C-terminal addition of a tag was expressed from the pMHC3×HA plasmid.

To exemplify, the following primers were used for the generation of phA4-3×HA (SEQ ID NOs: 1 and 2), an expression plasmid based on the pMHC3×HA plasmid and expressing the human APOBEC4 protein modified with three HA tags fused to the C-terminus: 5′ primer: cag gcg gta cca gcc tgg aga caa att gat gg (SEQ ID NO: 7), 3′ primer: gac ttg gat ccc ctt tct tcc ctt tct tct tct tct ttt cat ctg cct cct tgc tac (SEQ ID NO: 8).

For the generation of the pHA-hA4_cDNA3.1Zeo(+) expression plasmid (SEQ ID NOs: 3 and 4), based on the pcDNA3.1/Zeo(+) plasmid and expressing the human APOBEC4 protein modified with a HA tag fused to the N-terminus, the following primers were used: 5′ primer: cgg atc cct agc aat ggg ata tcc ata cga tgt tcc aga tta cgc tga gcc cat ata tga gga gta cc (SEQ ID NO: 9), 3′ primer: gaa ttc ttt att tct tcc ctt tct tct tct tc (SEQ ID NO: 10).

For the generation of the phA4_cDNA3.1Zeo(+) expression plasmid (SEQ ID NOs: 5 and 6), likewise based on the pcDNA3.1/Zeo(+) plasmid and expressing the human APOBEC4 protein, the following primers were used: 5′ primer cgg atc cct agc aat gga gcc cat ata tg (SEQ ID NO: 11), 3′ primer gaa ttc ttt att tct tcc ctt tct tct tct tc (SEQ ID NO: 12).

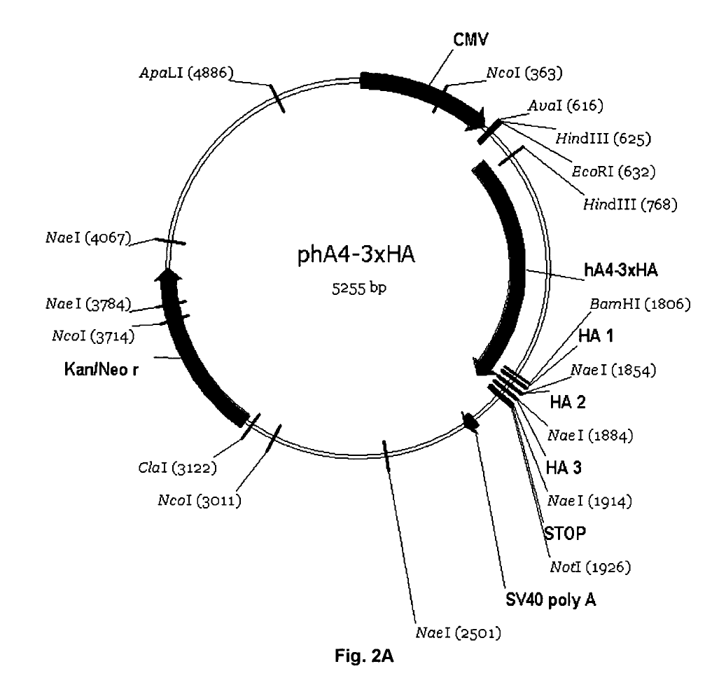

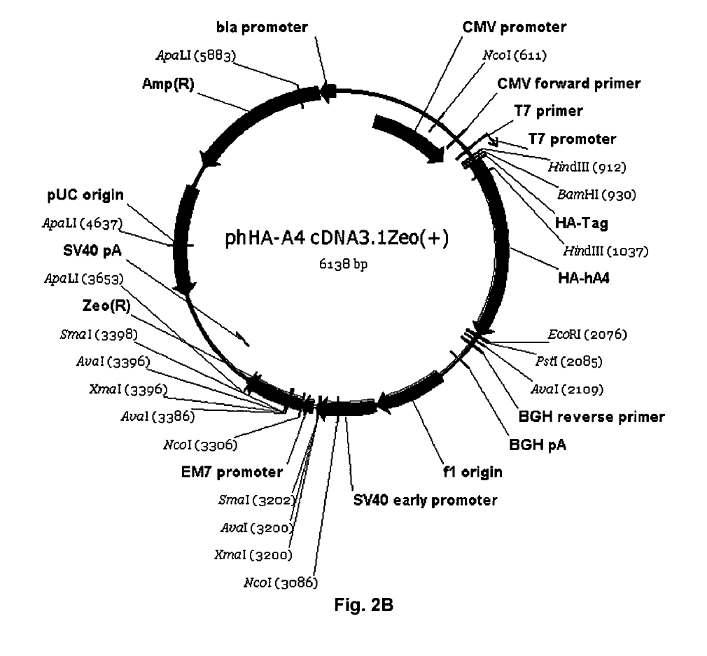

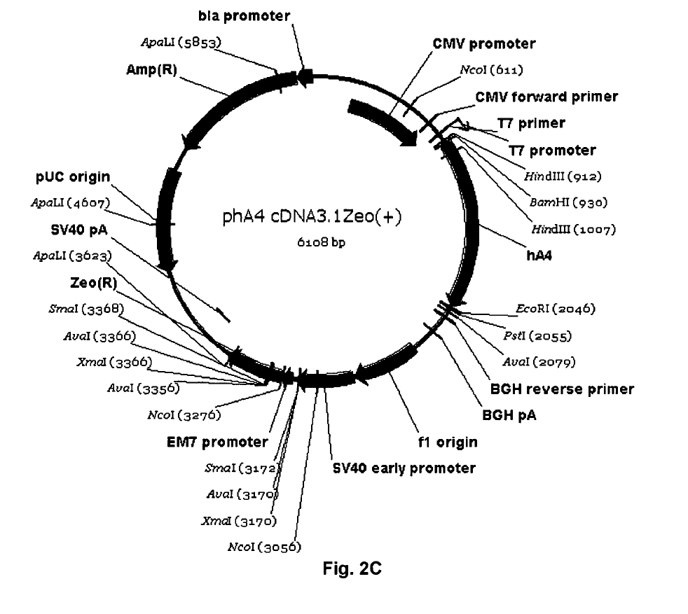

The amplicons generated were cloned into the respective plasmid vectors using the BamHI and EcoRI restriction enzymes. FIGS. 2A-C show plasmid maps of the three exemplified expression vectors phA4-3×HA, pHA-hA4_cDNA3.1Zeo(+), and phA4_cDNA3.1Zeo(+), respectively.

The skilled person will know, how to generate different expression vectors comprising, inter alia, different tags, for example the V5 or c-myc tag, a different number of tags, or how to introduce mutations into the APOBEC4 gene to, for example, truncate or completely delete the C-terminal poly-lysine tail, or insert mutations to zinc-finger domain.

### Cell Culture

In the following cell culture is exemplified for 293T cells. The use of different cells is envisaged and encompassed by the teaching of the present invention.

Adherend 293T cells were cultured in DMEM (GIBCO/BRL). For cell passage, cells were detached using a 1 mM EDTA/PBS solution and transferred into fresh medium (1:10) twice a week. Cells were washed using PBS (137 mM NaCl, 2.7 mM KCL, 4.3 mM Na₂HPO₄, 1.4 mM KH₂PO₄). All culturing media were supplemented with 10% fetal calf serum (FCS, Biochrom KG), 2 mM L-glutamine (Biochrom KG) and antibiotics (100 U/ml penicillin, 50 mg/ml streptomycine [Biochrome KG]). FCS was incubated for 30 min at 46° C. for inactivation of components of the complement system. Culturing of the cells was carried out in a cell incubator (BBD 6220, Heraeus) at 37° C. and 5% CO₂.

### Transfection and Production of Viral Cell Culture Supernatant

One day prior to transfection, 0.8×10⁶ 293T cells were seeded per well of cell culture plate. For transfection, LipofectamineLTX (Invitrogen) was used in a 2:1 ratio (μl LipofectamineLTX: μg plasmid DNA) according to the manufacturer's recommendations.

For the production of viral cell culture supernatant, cells were co-transfected with 1 μg plasmid DNA of a proviral genome, if required 0.5 μg expression vector pMD.G (expressing the VSV-G protein), and 0-3 μg of the respective APOBEC4 plasmid. If required, pcDNA3.1 plasmid was added to keep the total amount of DNA added at 4 μg. Harvesting of viral supernatant was carried out 48 hrs after transfection. Cell debris contained in the supernatants was removed by filtration using filters with a pore size of 45 μm (Sartorius). The filtrate was stored at −80° C.

### Luciferase Reporter Virus Test

Reporter viruses allow a fast and quantitative analysis of interactions of the cellular and viral components as well as of antiviral factors. To study the influence of APOBEC4 on the virus-host interaction the VSV-G pseudotyped reporter virus NL-luc R-E- was employed. NL-luc R-E- is derived from the HIV-1 variant NL4-3 and features a deletion of the env and nef genes. Furthermore, NL-luc R-E- carries the luciferase reporter gene of Photinus pyralis.

293T cells were transfected with equal amount of the different plasmids. 48 hrs after transfection and production of viruses/viral particles with or without the (modified) APOBEC4 of the present invention, viruses/viral particles were harvested and cell lysates (of about 2×10⁴ cells) were analyzed for luciferase expression using the Steadylite HTS Kit (Perkin Elmer) and according to the manufacturer's recommendations.

### Denaturing Polyacrylamide Gelelectrophoresis (SDS-PAGE) and Western Blot

For cell lysis, 293T cells were washed in 1 ml PBS after harvesting of the viruses/viral particles, detached using PBS/EDTA, pelletted (250 rpm, 5 min, RT) and lysed during an incubation of 5 min on ice after addition of RIPA lysis buffer (25 mM TRIS, pH 8, 137 mM NaCl, 1% glycerine, 0.1% SDS, 0.5% Na-deoxycholate, 1% NaPO₄). After a centrifugation step of 10 min at 14000 rpm and 4° C., protein concentration of the supernatant was carried out according to Bradford: 5 μl of the supernatant were added to 1000 μl of Bradford reagent (Bio-Rad, Hercules, USA) and the absorption at 595 nm was measured using a spectrometer (Gene Quant II, RNA/DNA-Calculator, Pharmacia Biotech).

For SDS-PAGE and western blotting, cell lysate corresponding to 30 μg of total protein was loaded to NuPAGE gradient gels (4-12%; Invitrogen) and electrophoresis was carried out according to the manufacturer'S recommendations. PVDF membrane (Invitrogene) was incubated with blocking solution (PBS, 5% milk powder, 0.5% Tween20 [Roth]) for 1 hr or over night at 4° C. to prevent an unspecific binding of the primary antibody. As primary antibodies, mouse anti-HA (1:5000 in blocking solution, MMS-101P, Covance), mouse anti-αTubulin (1:10000, B5-1-2, Sigma) or mouse anti-p24 (1:800, 183-H12-5C, The following reagent was obtained through the NIH AIDS Research and Reference Reagent Program, Division of AIDS, NIAID, NIH: HIV-1 p24 Monoclonal Antibody (183-H12-5C) from Dr. Bruce Chesebro and Kathy Wehrly). As secondary antibody, an anti-mouse horseradish peroxidase conjugated antibody (1:7500 GE Healthcare) was used. Before and after each incubation period, the membrane was first washed with washing solution 1 (PBS, 0.5% TWeen20) twice and subsequently incubated in washing solution 2 (PBS, 0.1% Tween20) for 25 min. Lumigen PS-3 Acridan (ECL plus Western Blotting Detection System, GE Healthcare) was used as the substrate, whose enzymatic conversion leads to light emission. Detection of the light emission was carried out using Amersham Hyperfilm ECL (GE Healthcare) in developing solution (Curix 60, Agfa).

### Example 1

### Modulation of HIV-1 NL4-3 Virus Production by APOBEC4 Expression

In the following, 293T cells are used as an exemplary cell type and HIV-1 is used as an exemplary virus.

293T cells were co-transfected as described above with an expression vector comprising the proviral nucleic acid sequence of the HIV-1 variant HIV-1 NL4-3 and expression vectors coding for the unmodified human APOBEC4 protein, as well as modified human APOBEC4 proteins HA-APOBEC4, APOBEC-HA and APOBEC4-3×HA, respectively. Subsequently, production of viruses in the viral supernatant (V) and cell lysates (C), respectively was determined in a reverse transcriptase assay (RT-PCR; not shown) and western blotting. Specifically, expression of the HIV antigen p24 and the HA tag and tubulin was assessed.

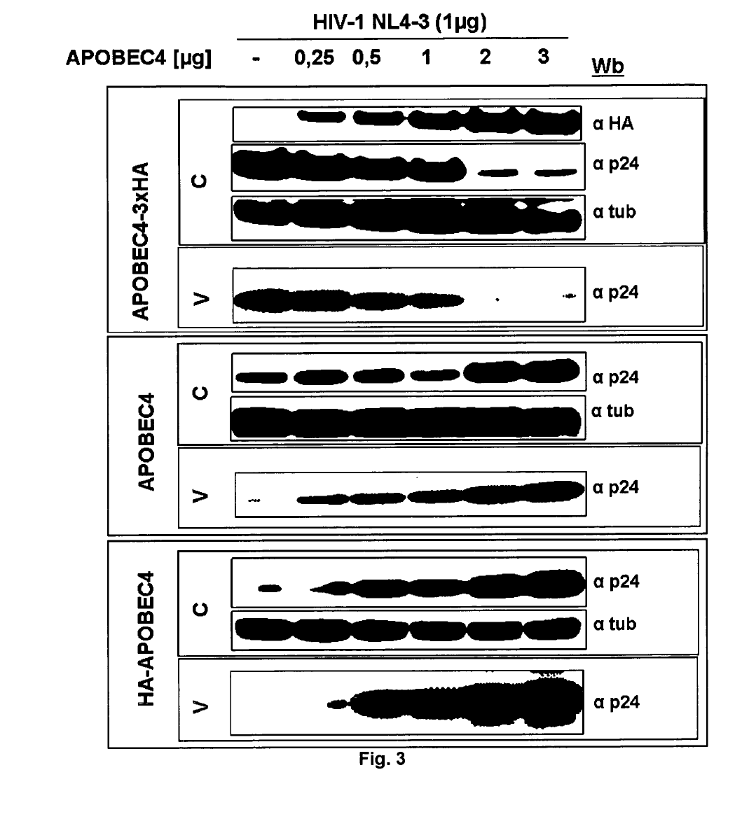

FIG. 3 shows the results of the western blotting. Co-transfection of the cells with HIV-1 NL4-3 and the expression plasmid coding for the unmodified APOBEC4 protein lead to an increase in viral production in both viral supernatant (V) and cell lysates (C) that directly and linearly correlated to the amount of APOBEC4 plasmid used during co-transfection (FIG. 3, middle panel).

Co-transfection with the expression plasmid coding for HA-APOBEC4 protein lead to an augmented increase in viral production in both viral supernatant and cell lysates compared to co-transfection with unmodified APOBEC4 protein. Viral production that resulted in co-transfection with HA-APOBEC4 expression plasmid was increased 5-10-fold compared to that of co-transfection with APOBEC4 expression plasmid. Likewise, the amount of produced viruses directly and linearly correlated to the amount of HA-APOBEC4 plasmid used during co-transfection (FIG. 3, bottom panel).

Co-transfection with the expression plasmid coding for APOBEC4-3×HA protein lead to a drastic decrease (up to 95%) in viral production in both viral supernatant and cell lysates compared to co-transfection with unmodified APOBEC4 protein. Likewise, the degree of inhibition of virus production directly and linearly correlated to the amount of APOBEC4-3HA plasmid used during co-transfection (FIG. 3, top panel). Similar results were obtained using an expression plasmid coding for APOBEC4-HA (not shown).

Together the results indicate that an APOBEC4 complex is recruited by the virus-infected cell during virus production. If additional APOBEC4 protein is present, virus production is increased. Expression of a modified APOBEC4 protein that is stabilized by modification of the N-terminus, inter alia, by addition of an amino acid sequence, for example, the HA tag, leads to an increased amount of APOBEC4 complex in the cell and thus an increased production of the virus by said cell. Expression of a modified APOBEC4 protein whose binding to the C-terminal poly-lysine tail is decreased or inhibited, inter alia, by modification of the C-terminus by, for example addition of an amino acid sequence as the HA tag or three HA tags, leads to a decreased amount of APOBEC4 complex in the cell and thus a decreased production of viruses by said cell.

Furthermore, and as evident from the results of, e.g., the western blotting, the pro- and anti-viral effects, respectively according to the present invention directly correlate with the amount of APOBEC4 expression vector used for transfection. In other words, the ration between expression vector coding for the proviral genome and plasmid vector encoding the (modified) APOBEC4 protein determines the level of increase or decrease of viruses by the transfected cell. As the amount of DNA that can be transfected into a cell is limited due to the cytotoxicity of the transfection reagent, cell lines stably expressing the (modified) APOBEC4 protein were generated and subsequently transfected with expression vectors encoding various proviral genomes. In such cell lines the pro- and anti-viral effects of the present invention were clearly enhanced.

### Example 2

### Modulation of HIV-1 NL-luc R-E- Virus Production by APOBEC4 Expression

The experiments of Example 1 were repeated using the HIV-1 NL4-3 derivate NL-luc R-E-, pseudotyped with the VSV-G protein.

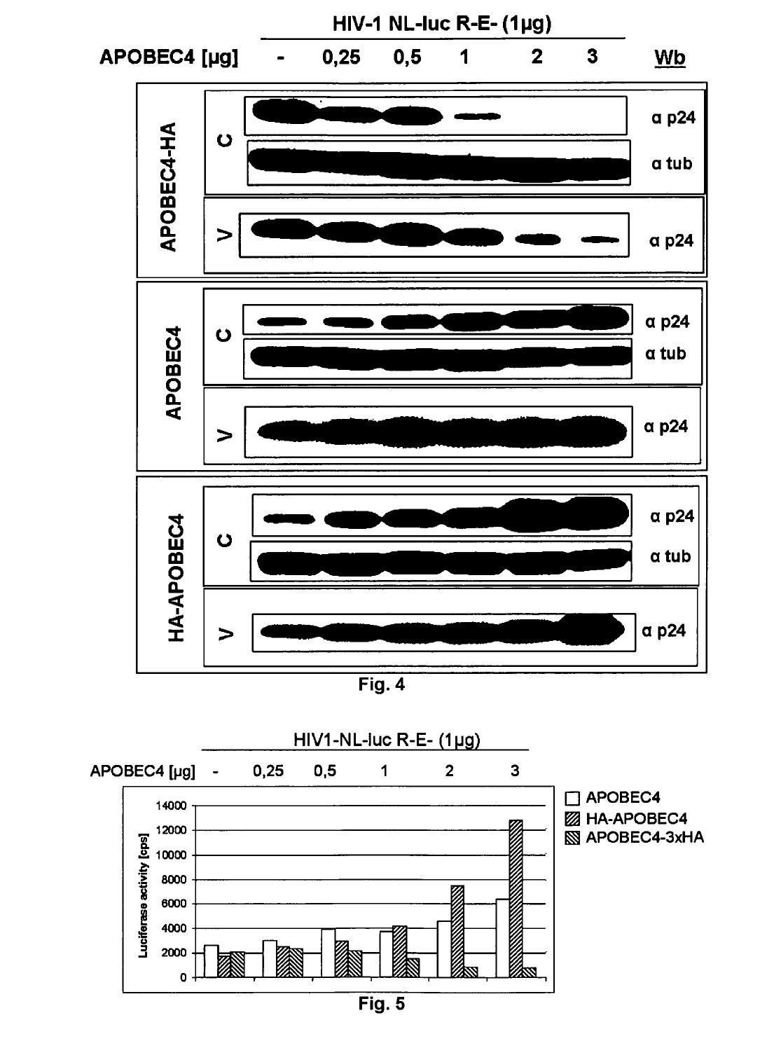

FIG. 4 shows the results of the western blotting that confirm the findings from Example 1. Co-transfection of the cells with an expression plasmid encoding the reporter virus and the expression plasmid coding for the unmodified APOBEC4 protein lead to an increase in viral production in both viral supernatant and cell (FIG. 4, middle panel); co-transfection with the expression plasmid coding for HA-APOBEC4 protein lead to an augmented increase in viral production (FIG. 4, bottom panel) and co-transfection with the expression plasmid coding for APOBEC4-3×HA protein lead to a drastic decrease in viral production (FIG. 4, top panel). Likewise, the degree of increase or decrease in virus production directly and linearly correlated to the amount of APOBEC4 expression plasmid used during co-transfection. Similar results were obtained using an expression plasmid coding for APOBEC4-HA (not shown).

Together the results indicate that the pro- and antiviral effects disclosed by the present invention are not restricted to wild type proviral genomes but are also applicable to, for example, pseudotyped viruses.

### Example 3

### Reporter Virus Assays

The VSV-G pseudotyped reporter virus NL-luc R-E- was employed to verify the results determined by western blotting. 293T cells were transfected as described above, viruses were harvested and cell lysates of about 2×10⁴ cells were analyzed for luciferase expression.

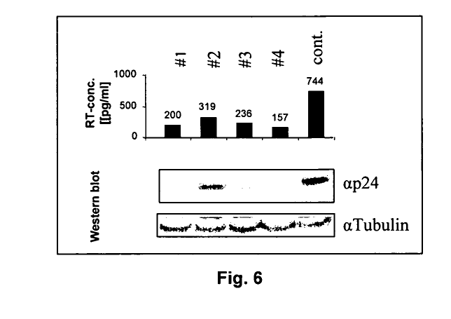

FIG. 5 shows the results of the reporter virus assays and demonstrates that the expression of the luciferase reporter gene (instead of the viral nef gene) depends on APOBEC4 expression in the same manner as the production of viruses depends on APOBEC4 expression. Co-transfection of the cells with NL-luc R-E- and the expression plasmid coding for the unmodified APOBEC4 protein lead to an increase in luciferase activity that directly and linearly correlated to the amount of APOBEC4 plasmid used during co-transfection.

Co-transfection with the expression plasmid coding for HA-APOBEC4 protein lead to an augmented increase in luciferase activity compared to co-transfection with unmodified APOBEC4 protein. Likewise, the increase in luciferase activity directly and linearly correlated to the amount of HA-APOBEC4 plasmid used during co-transfection.

Co-transfection with the expression plasmid coding for APOBEC4-3×HA protein lead to a drastic decrease in luciferase activity compared to co-transfection with unmodified APOBEC4 protein. Again, the degree of decrease in luciferase activity directly and linearly correlated to the amount of APOBEC4-3HA plasmid used during co-transfection. Similar results were obtained using an expression plasmid coding for APOBEC4-HA (not shown).

Together these results demonstrate that the reporter virus assays of the present invention allow for a fast and quantitative analysis of interactions of the cellular and viral components as well as of antiviral factors. Furthermore, the results demonstrate that the pro- and anti-viral effects of the present invention are also applicable for viruses that are genetically modified, for example, to carry a heterologous gene.

### Example 4

### Modulation of Production of Different Virus Strains by APOBEC4 Expression

The experiments of the foregoing Examples were repeated using a truncated virus genome coding only for the structural proteins and enzymes (Gag, Pol). Furthermore, SIVagm, SIVmac and MLV were tested as additional different virus strain.

For all tested viruses the results of the foregoing Examples could be verified. In each case, co-transfection of the cells with an expression plasmid encoding the proviral genome and the expression plasmid coding for the unmodified APOBEC4 protein lead to an increase in viral production in both viral supernatant and cell; co-transfection with the expression plasmid coding for HA-APOBEC4 protein lead to an augmented increase in viral production and co-transfection with the expression plasmid coding for APOBEC4-3×HA or APOBEC3-HA protein lead to a drastic decrease in viral production. In each case, the degree of increase or decrease in virus production directly and linearly correlated to the amount of APOBEC4 expression plasmid used during co-transfection.

Together the results indicate that the pro- and antiviral effects disclosed by the present invention are not restricted to the HIV-1 virus but are also applicable to, for example, truncated HIV viruses and/or various other retroviruses or lentiviruses. Therefore, the present invention provides means to increase or decrease, i.e. modulate the production of retroviruses and lentiviruses or particles thereof.

### Example 5

### APOBEC4 as a Target for Antiretroviral Drugs

293T cells or HeLa cells are contacted with a candidate compound and the amount of APOBEC4 transcript and/or APOBEC4 protein is determined by RT-PCR, Northern blotting and western blotting, respectively. A decrease of the APOBEC4 transcript and/or APOBEC4 protein in the presence of the candidate compound as compared to the absence of the candidate compound indicates that the candidate compound is a potential inhibitor of APOBEC4 transcription and/or expression and thus a potential antiretroviral drug.

In another assay, 293T or HeLa cells infected with a retrovirus are contacted with one or more test compound, for example siRNA or shRNA molecules directed against APOBEC4 mRNA, and the titer of the retrovirus is determined in the presence and the absence of a test compound. A decrease in the titer indicates that the test compound interferes with the assembly of the retrovirus by interaction with the APOBEC4 protein and thus is a potential antiretroviral drug.

### Example 6

### Therapeutic Downregulation of Endogenous APOBEC4

One day before transfection 0.8×10⁶ 293T cells were seeded in 6well-plates. For co-transfection with 1 μg expression vector comprising the proviral nucleic acid sequence of the HIV-1 variant HIV-1 NL4-3 and 0.3 μg vectors coding for the siRNA targeting sequences within the 3′ untranslated region of APOBEC4 mRNA the HiPerFect Transfection Reagent (Qiagen) was used following manufacturer's recommendation. Negative control siRNA (QIAGEN) was also transfected (0.3 μg). The following siRNAs were used:

| siRNA | Sense sequence |
|---|---|
| Hs_MGC26594_1 SI00636307 | CAGCATGAAACCAATTACCAA |
| Hs_MGC26594_2 SI00636314 | AAGGCTGAAATGACATCTCAA |
| Hs_MGC26594_3 SI00636321 | TAGGAAGTGCTCCTTGATTAA |
| Hs_MGC26594_4 SI00636328 | TACCATATCAACTATTATAAA |
| Negative Control siRNA′ | UUCUCCGAACGUGUCACGUdTdT |

Two days after transfection virus particles were harvested and cell lysates were generated as described. Production of viruses in the viral supernatant and cell lysates, respectively was determined in a reverse transcriptase assay and western blotting. Specifically, expression of the HIV antigen p24 and tubulin was assessed. The results depicted in FIG. 6 show that after knock down of APOBEC4 by siRNA the virus production (as measured by the concentration of released virus; RT values) was drastically reduced. This finding correlates with reduction of the intracellular level of the viral protein p24 after knock down of APOBEC4 mRNA as shown by the western blot in FIG. 6. Together these findings demonstrate that the downregulation of endogenous APOBEC4 directly leads to a decreased virus production and thus provides a suitable treatment for retroviral infections.

### REFERENCES

ROGOZIN, I B, et al. APOBEC4, a new member of the AID/APOBEC family of polynucleotide (deoxy)cytidine deaminases predicted by computational analysis. Cell Cycle. 2005, vol. 4, no. 9, p. 1281-5.
CONTICELLO, S G, et al. Evolution of the AID/APOBEC family of polynucleotide (deoxy)cytidine deaminases. Mol Biol Evol. 2005, vol. 22, no. 367, p. 77.
KIM, V N, et al. Minimal requirement for a lentivirus vector based on human immunodeficiency virus type 1. J. Virol. 1998, vol. 72, no. 1, p. 811-6.
ALTSCHUL, S F, et al. Basic local alignment search tool. J Mol Biol. 1990, vol. 215, no. 3, p. 403-10.
PERASON, W R, et al. Improved tools for biological sequence comparison. Proc Natl Acad Sci USA. 1988, vol. 85, no. 8, p. 2444-8.
VIGNA, E, et al. Lentiviral vectors: excellent tools for experimental gene transfer and promising candidates for gene therapy. J Gene Med. 2000, vol. 2, no. 5, p. 308-16.
PALU, G, et al. Progress with retroviral gene vectors. Rev Med Virol. 2000, vol. 10, no. 3, p. 185-202.

1. A modified APOBEC4 protein characterized in that the N-terminus is modified to stabilize the APOBEC4 protein or the C-terminus is modified to decrease or inhibit binding to the C-terminal poly-lysine tail of APOBEC4.

2. The modified APOBEC4 protein according to claim 1, wherein the modification of the N-terminus comprises addition of at least one amino acid sequence selected from the group consisting of the V5 tag, the c-myc tag and the HA tag or an amino acid sequence homologous thereto.

3. The modified APOBEC4 protein according to claim 2, wherein the at least one amino acid sequence is HA or at least 90%, 95%, 97%, 99%, or 99.5% identical thereto.

4. The modified APOBEC4 protein according to claim 1, wherein the modification of the C-terminus comprises deletion or substitution of at least one amino acid of the poly-lysine tail or comprises addition of at least one amino acid sequence selected from the group consisting of the V5 tag, the c-myc tag and the HA tag or an amino acid sequence homologous thereto.

5. The modified APOBEC4 protein according to claim 4, wherein the at least one amino acid sequence is HA or 3 HA or at least 90%, 95%, 97%, 99%, or 99.5% identical thereto.

6. A cell expressing the modified APOBEC4 protein according to claims 1-5.

7. The cell according to claim 6, wherein the cell further produces a retrovirus or viral particles thereof.

8. The cell according to claim 7, wherein the retrovirus is a lentivirus and/or is a replication deficient virus.

9. The cell according to claim 8, wherein the N-terminus of the APOBEC4 protein is modified to stabilize the APOBEC4 protein.

10. The cell according to claims 6-9, wherein the cell is selected from the group consisting of a 293T cell, a HeLa cell, a T-helper cell, a macrophage and a stem cell or progenitor cells or cell lines thereof.

11. A nucleic acid coding for the APOBEC4 protein according to anyone of claims 1-5 or the complement thereof.

12. A nucleic acid comprising the nucleic acid sequence according to claim 11.

13. A nucleic acid or vector for gene therapy comprising the nucleic acid according to claim 12, wherein the nucleic acid coding for APOBEC4 is operably linked to a promoter.

14. A vaccine comprising the cell according to claim 9 and an acceptable pharmaceutical carrier.

15. A retroviral replication deficient particle comprising the nucleic acid according to claims 12-13, wherein the nucleic acid is an RNA.

16. The retroviral particle according to claim 15, wherein the retroviral particle is derived from HIV, HIV-1, HIV-2, SIV, SIVagm, SIVmac, spuma virus, FIV, EIAV or MLV.

17. A method for decreasing the production of retroviruses or lentiviruses or viral particles thereof of a cell producing a retrovirus, lentivirus or vector particles thereof, the method comprising expressing a modified APOBEC4 protein according to claims 1, 4 and 5 in said cell, wherein the C-terminus of the APOBEC4 protein is modified to decrease or inhibit binding to the C-terminal poly-lysine tail of APOBEC4.

18. The method according to claim 17, wherein the production of retroviruses or viral particles thereof is decreased by at least 20%.

19. A method for producing retroviral particles, the method comprising cultivating a cell according to claim 9.

20. The use of a cell according to claim 9, the nucleic acid according to claim 13 or the retroviral particle according to claims 15-16 for the manufacturing of a medicament.

21. The use of a cell according to claim 9, the nucleic acid according to claim 13 or the retroviral particle according to claims 15-16 for the manufacturing of a medicament for the treatment of or vaccination against an infection by a retrovirus.

22. A method for screening for antiviral compounds, the method comprising the steps of:
contacting a cell expressing an APOBEC4 protein with a test compound; and
determining whether the test compound affects APOBEC4 transcription and/or expression of said cell,
wherein a decrease in APOBEC4 transcription and/or expression of said cell indicates that the test compound is an antiviral compound.

23. The method according to claim 22, wherein the APOBEC4 transcription and/or expression of a first cell in the presence of a test compound is compared to the APOBEC4 transcription and/or expression of a second cell in the absence of said test compound,
wherein a decrease in APOBEC4 transcription and/or expression of the first cell compared to the second cell indicates that the test compound is an antiviral compound.

24. The method according to claim 22 or 23, wherein the APOBEC4 transcription and/or expression is determined by RT-PCR, Northern blotting and/or western blotting.

25. A method for screening for antiviral compounds, the method comprising the steps of:
contacting a cell expressing a retrovirus and an APOBEC4 protein with a test compound; and
determining the titer of the retrovirus in the presence and the absence of the test compound,
wherein a decrease in the titer indicates that the test compound is an antiviral compound.

26. The method according to anyone of claims 22-25, wherein the test compound is an siRNA or shRNA molecule directed against APOBEC4 mRNA.

27. Method for the treatment of retroviral infections, comprising the step of downregulating or depleting of endogenous APOBEC4.

28. Method according to claim 27, wherein the downregulation is achieved by administration of at least one siRNA and/or at least one shRNA directed to the APOBEC4 gene.

29. Method according to claim 27 or 28, wherein the depletion is achieved by administration of at least one antibody directed to APOBEC4.

30. An siRNA, shRNA and/or an antibody as a medicament, wherein the siRNA and shRNA is directed to the APOBEC4 mRNA and the antibody is directed to APOBEC4.

31. The siRNA and/or shRNA according to claim 30, wherein the siRNA and/or shRNA is directed to the 3′ untranslated region of APOBEC4 mRNA.

32. An siRNA, shRNA and/or an antibody for the treatment of retroviral infections, wherein the siRNA and shRNA is directed to the APOBEC4 mRNA and the antibody is directed to APOBEC4.
